(19) United States
(12) Patent Application Publication
(10) Pub. No.: US 2020/0315740 A1
(43) Pub. Date: Oct. 8, 2020

(54) IDENTIFICATION AND ASSIGNMENT OF INSTRUMENTS IN A SURGICAL SYSTEM USING CAMERA RECOGNITION

(71) Applicant: TransEnterix Surgical, Inc., Morrisville, NC (US)

(72) Inventors: Nicholas J Jardine, Holly Springs, NC (US); Matthew Robert Penny, Holly Springs, NC (US); Caleb T Osborne, Durham, NC (US); Bruce Wiggin, Raleigh, NC (US); Kevin Andrew Hufford, Cary, NC (US)

(21) Appl. No.: 16/732,303

(22) Filed: Dec. 31, 2019

Related U.S. Application Data
(60) Provisional application No. 62/787,247, filed on Dec. 31, 2018.

(51) Int. Cl. A61B 90/90; A61B 34/30; A61B 1/04; A61B 1/313; A61B 90/92; A61B 90/96; A61B 90/00

(52) U.S. Cl. CPC A61B 90/90; A61B 34/30; A61B 1/04; A61B 1/3132; A61B 17/29; A61B 90/96; A61B 90/361; A61B 2034/302; A61B 90/92

(57) ABSTRACT

In a surgical system in which surgical instruments are manipulated using robotic arms that move in response to user input from a plurality of input devices. Images capturing an instrument positioned within view of a camera are received. At least one characteristic of the surgical instrument is determined by the system based on the image data. The system automatically assigns the surgical instrument to a user input device, or controls movement of the robotic manipulator carrying the instrument, based on the at least one characteristic. The surgical instrument is then robotically maneuvered by the corresponding robotic arm based on user input to the user input device.

### TECHNICAL FIELD OF THE INVENTION

The present invention relates generally to the use of camera recognition is surgery, and more specifically to the use of camera recognition to determine type, characteristics or other features of instruments being used in surgery

### BACKGROUND

There are various types of surgical robotic systems on the market or under development. Some surgical robotic systems use a plurality of robotic arms. Each arm carries a surgical instrument, or the camera used to capture images from within the body for display on a monitor. See U.S. Pat. No. 9,358,682 and US 20160058513. Robotic systems use motors to position and/or orient the camera and instruments and to, where applicable, actuate the instruments. Typical configurations allow two or three instruments and the camera to be supported and manipulated by the system. Input to the system is generated based on input from a surgeon positioned at a surgeon console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

Although the concepts described herein may be used on a variety of robotic surgical systems, one robotic surgical system is shown in FIG. 1. In the illustrated system, a surgeon console 12 has two input devices such as handles 17, 18. The input devices 12 are configured to be manipulated by a user to generate signals that are used to command motion of a robotically controlled device in multiple degrees of freedom. In use, the user selectively assigns the two handles 17, 18 to two of the robotic manipulators 13, 14, 15, allowing surgeon control of two of the surgical instruments 10a, 10b, and 10c disposed at the working site (in a patient on patient bed 2) at any given time. To control a third one of the instruments disposed at the working site, one of the two handles 17, 18 may be operatively disengaged from one of the initial two instruments and then operatively paired with the third instrument, or another form of input may control the third instrument as described in the next paragraph. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 17, 18 are configured to be manipulated by a user to generate signals that are processed by the system to generate instructions used to command motion of the manipulators in order to move the instruments in multiple degrees of freedom and to, as appropriate, control operation of electromechanical actuators/motors that drive motion and/or actuation of the instrument end effectors.

The surgical system allows the operating room staff to remove and replace the surgical instruments 10a, b, c carried by the robotic manipulator, based on the surgical need.

When an instrument exchange is necessary, surgical personnel remove an instrument from a manipulator arm and replace it with another.

Some surgical and industrial robotic systems are configured to interchangeably receive a variety of end effectors. Different end effectors might possess different dimensions, geometry, weight characteristics, shaft length, jaw open-close ranges, etc. Some instrument may have no articulating features, others may be controlled to articulate in multiple degrees of freedom. Some may have jaws and others do not. For these reasons, when an end effector is mounted to a robotic manipulator, the system can most optimally move and actuate the end effector if the system has been given input as to the characteristics of the end effector. This application describes a system and method for giving input to the surgical robotic system relating to the type of end effector that has been mounted.

### BRIEF DESCRIPTION OF THE DRAWINGS

FIG. 1 is a perspective view of one type of surgical robotic system.

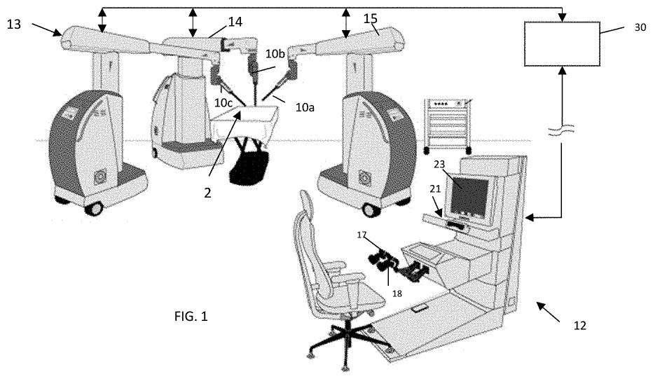

FIG. 2 shows a camera image display used to facilitate tool assignment.

### DETAILED DESCRIPTION

This application describes use the laparoscopic camera to identify instruments and communicate that back to the surgeon console. Additionally, the method may allow for automatic assignment of the instrument to the manipulator arm via recognition of which side of the screen the instrument enters.

A control unit provided with the surgical system includes a processor able to execute programs or machine executable instructions stored in a computer-readable storage medium (which will be referred to herein as “memory”). Note that components referred to in the singular herein, including “memory,” “processor,” “control unit” etc. should be interpreted to mean “one or more” of such components. The control unit, among other things, generates movement commands for operating the robotic arms based on surgeon input received from the input devices at the surgeon console.

The memory includes computer readable instructions that are executed by the processor to perform the methods described herein. These include methods of using image input from the laparoscopic camera to identify and recognize eye tracking input in a sequence for assigning user input devices to selected surgical instruments via their shape, color (IR spectrum, visual or patterns), or markings (QR codes, etched or other laser markings, bar code) camera (detection of shapes, colors, markings).

In use, after an instrument has been positioned such that an image of its relevant features can be captured by the camera, the processor receives image data input based on the captured images of the instrument. The image may be captured outside the body or inside the body. It may be captured before or after the instrument is mounted to the robotic arm. Outside the body, an image of the end effector of the instrument may be captured before the instrument is inserted into the body using the laparoscopic camera or another camera located outside the body, or an image of a more proximal part of the instrument may be captured outside the body (regardless of whether the end effector has been inserted into the body) using the laparoscopic camera before that camera is positioned inside the body, or using an externally positioned camera. Inside the body, image capture for instrument recognition may be performed using the laparoscopic camera or auxiliary image sensors (which may be broadly referred to using the term “camera”) used for computer vision applications.

After receiving image data, the processor determines or derives information about the instrument using, for example, other information stored in the memory. This information can include a correlation of the image data (i.e. data relating to the instrument shape, etched markings, QR code, color etc.) to the instrument type and/or to specific instrument geometric properties or drive parameters for that instrument. The system may then control operation of the instrument using the appropriate drive parameters for the instrument type. The system may also automatically assign the instrument to one of the user input devices based on the determined instrument type. For example, if the surgical procedure to be performed is one to be carried out with an instrument of type 1 controlled by the user's right hand and an instrument of a second type, type 2, controlled by the user's left hand, the processor, upon receiving image data indicating that a type 1 instrument had been mounted to a surgical manipulator, may automatically assign that instrument to the right hand user input device. In another embodiment, different regions may be displayed on the visual display.

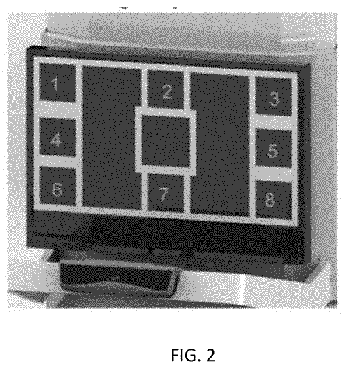

These regions could be used to automatically assign an instrument to the right/left user input device. An example of such an image display is shown in FIG. 2.

1.-2 (canceled)

3. A robotic surgical system, comprising:
a surgical instrument moveable by a robotic manipulator within a work area;
a camera positioned to capture an image of a portion of the surgical instrument;
a processor configured to determine, based on the image data from the sensor, at least one characteristic of the surgical instrument and to cause robotic control of the surgical instrument based on the determined characteristic.

4. The system of claim 3, wherein the camera is positioned in the work area inside a patient's body cavity to capture an image of a portion of the surgical instrument inside the body cavity.

5. The system of claim 4, wherein the system further includes an image display and wherein the camera is positioned to capture an image of the work area for display on the image display.

6. The system of claim 4, wherein the system further includes an image display, and wherein the system includes a second camera positioned to capture an image of the work area for display on the image display

7. The system of claim 3, wherein the camera is positioned to capture an image of a portion of the surgical instrument outside the patient's body cavity.

8. The system of claim 3 wherein the characteristic pertains to a shape, color, color pattern, marking, texture, QR code, bar code or other code on the instrument.

9. The system of claim 3 wherein the characteristic is an instrument type, length, geometry, mass or weight, jaw open-close range, jaw type, or an identification of operable degrees of freedom.

10. A method of robotically controlling a surgical instrument, comprising the steps of:
receiving image data for an instrument positioned within view of a camera;
determining, based on the image data, at least one characteristic of the surgical instrument; and
robotically controlling the surgical instrument based on the at least one characteristic.

11. The method of claim 10, wherein the method includes capturing the image data using a camera positioned in a work area inside a patient's body cavity.

12. The method of claim 11, wherein the method further displaying images from the camera on an image display.

13. The method of claim 11, wherein the camera is a first camera and wherein the method further includes capturing second images of the work area using a second camera and displaying the second images, but not the images from the first camera, on an image display.

14. The method of claim 10, wherein the method includes capturing the images of a portion of the surgical instrument outside the patient's body cavity.

15. The method of claim 10 wherein the characteristic pertains to a shape, color, color pattern, marking, texture, QR code, bar code or other code on the instrument.

16. The method of claim 10, wherein the characteristic is an instrument type, length, geometry, mass or weight, jaw open-close range, jaw type, or an identification of operable degrees of freedom.

17. A method of robotically controlling a surgical instrument, comprising the steps of:
receiving image data for an instrument positioned within view of a camera;
determining, based on the image data, at least one characteristic of the surgical instrument; and
automatically assigning the surgical instrument to a user input device based on the at least one characteristic; and
robotically controlling the surgical instrument based on user input to the user input device.

18. The method of claim 17, wherein the method includes capturing the image data using a camera positioned in a work area inside a patient's body cavity.

19. The method of claim 17, wherein the method includes capturing the images of a portion of the surgical instrument outside the patient's body cavity.

20. The method of claim 17 wherein the characteristic pertains to a shape, color, color pattern, marking, texture, QR code, bar code or other code on the instrument.

21. The method of claim 17, wherein the characteristic is an instrument type, length, geometry, mass or weight, jaw open-close range, jaw type, or an identification of operable degrees of freedom.
